(19) United States
(12) Patent Application Publication
(10) Pub. No.: US 2026/0335567 A1
(43) Pub. Date: Oct. 29, 2026

(54) HIGH-PRESSURE MEDICAL PUMPING DEVICE AND INHALATION DEVICE

(71) Applicant: invoX Belgium NV, Diepenbeek (BE)

(72) Inventors: Jürgen Rawert, Cologne (DE); Steven Madden, Hamburg (DE)

(21) Appl. No.: 19/521,390

(22) PCT Filed: Oct. 4, 2024
(86) PCT No.: PCT/EP2024/077937

§ 371 Date: Mar. 20, 2026

(30) Foreign Application Priority Data
Oct. 4, 2023 (EP) 23201555.2
Oct. 4, 2023 (EP) 23201557.8

(51) Int. Cl. A61M 11/00

(52) U.S. Cl. CPC A61M 11/007

(57) ABSTRACT

The invention provides a high-pressure medical pumping device for providing discrete portions of a medical fluid in pressurized form, the pumping device comprises ⋅—a fluid inlet; ⋅—a fluid outlet; and ⋅—a pumping unit fluidically connecting the fluid inlet with the fluid outlet and being adapted to generate the discrete portions of the medical fluid in pressurized form and to convey the discrete portions of the medical fluid (in a downstream direction) from the fluid inlet to the fluid outlet; wherein the pumping unit comprises ⋅—a pump cylinder (9) comprising a pump chamber (3) located within the pump cylinder comprising a fluid opening, wherein the pump chamber has a variable chamber height (CH); and ⋅—a longitudinal piston (5) comprising a first end and a second end located opposite of the first end, wherein the longitudinal piston is at least partly located within the pump chamber of the pump cylinder such that at least the first end of the piston is located within the pump chamber, and wherein the piston and the pump cylinder are arranged for back and forth stroke movement of at least one of the piston and the pump chamber relative to each other, wherein the piston is of circular shape with an outer diameter (OD), ⋅—means (7) to exert a release force (RF) to initiate a stroke movement of the piston and/or the pump chamber to convey the fluid to the fluid outlet; wherein the piston comprises a fluid channel fluidically connecting the first end of the piston with the second end of the piston and being adapted to convey the medical fluid from the first or second end of the piston to the corresponding opposite end of the piston, wherein said fluid channel has a diameter corresponding to an inner diameter (ID) of the piston; and wherein the pumping unit is adapted to provide the discrete portions of the medical fluid in pressurized form at a pressure of at least 100 bar.

### FIELD OF THE INVENTION

The invention relates to the field of pumping devices for medical devices, specifically inhalation devices for medical liquids for inhalation therapy. In particular, the invention relates to a to a high-pressure medical pumping device for providing discrete portions of a medical fluid in pressurized form, the pumping device comprising a pump unit having a pump cylinder and a piston arranged for back-and forth stroke movement within the pump cylinder.

### BACKGROUND OF THE INVENTION

Pumping devices or units for medical devices, specifically for pumping medical fluids are known from the art since a long time ago. For example, such devices are known from inhalation devices for the application of medical fluids or active ingredients in the form of aerosols, i.e., small liquid droplets embedded in a gas. Such an inhalation device is known, e.g., from document WO 91/14468 A1. Essential components of this inhalation device are a reservoir in which the liquid that is to be aerosolized is contained; a pumping device for generation of a pressure being sufficiently high for nebulizing; as well as an atomizing device in the form of a nozzle. By means of the pumping device, the liquid is drawn in a discrete amount, i.e., not continuously, from the reservoir, and fed to the nozzle. The pumping device works without propellant and generates pressure mechanically.

The document further discloses an embodiment in which the pressure in the pumping chamber which is connected to the housing is generated by movement of a moveable hollow piston. The piston is moveably arranged inside the immobile cylinder or pumping chamber. The (upstream arranged) inlet of the hollow piston is fluidically connected to the interior of the reservoir (reservoir pipe section). Its (downstream arranged) tip leads into the pumping chamber. Furthermore, a check valve that inhibits a back flow of liquid into the reservoir is arranged inside the tip of the piston.

For filling the piston, the same is directly connected with its upstream end to the reservoir. By pulling out the piston of the pumping chamber provided inside a hollow cylinder, its interior volume is enlarged, such that an increasing under pressure is built up inside the pumping chamber. This pressure propagates through the hollow piston into the reservoir, such that liquid is sucked from the same into the piston. At the same time, said valve opens at its tip, since the pressure inside the reservoir is higher than inside the (yet empty) pumping chamber. The pumping chamber is being filled. At the same time, a spring is loaded and locked at the motion's end when the moveable piston has reached its lower dead center (in the case of a vertically arranged device) and the pumping chamber is filled.

A further example of a pumping device for the generation of discrete amounts of a medical fluid in pressurized form for inhalation is disclosed in US 2014/0076308 A1. The document discloses a specific inhaler having a pumping unit with a pressure chamber and a conveying tube acting as a piston within the pressure chamber. The conveying tube has nonreturn valve positioned at its the downstream end located within the pressure chamber.

The piston is arranged inside the pressure spring, which is designed as helical spring, thus limiting its outside diameter. Also because of the typically small volume (e.g., 15 μl), the piston is designed with a thin interior (and often also exterior) diameter.

This typically small inner diameter of the conveying tube or, in other words, moveable piston of, e.g., 0.3 to 1.0 mm, together with a small size of the check valve being arranged within, is a drawback of the described construction. The small diameter results in a high flow resistance, such that in particular, media of higher viscosities flow into and through the piston only very slowly. In other words, the described construction is suitable especially for low-viscosity (aqueous) liquids and for emitting low doses thereof. Furthermore, fabrication of a sufficiently tight check valve of small diameter is difficult.

An improved inhalation device utilizing a non-moveable piston is disclosed in WO 2018/197730 A1. The device overcomes some of the drawbacks of the above-described prior art.

Furthermore, the pumping units of the above-described devices are designed for generating discrete portions of the medical fluid to be aerosolized at elevated pressures necessary for the aerosolization of the respective medical fluid, often in the range of about 10 to about 100 MPa and above.

Devices of the prior art provide small volumes of fluid (e.g., about 15 μL) with their pump cycle, due to the limitations of the devices. Larger volumes (e.g., 25 μL) result in increased mechanical stress of the devices or a highly increased spray time of the fluid, which is inconvenient for the user and may result in reduced patient compliance. If the device is able to release a larger amount of fluid the quality of the generated aerosol is often not ideal for inhalation.

It is thus an object of the present invention to provide for an improved high-pressure medical pumping device for providing discrete portions of a medical fluid in pressurized form that overcomes the at least some of the above-discussed drawbacks of the prior art devices. Further objects of the invention will be clear on the basis of the following description of the invention, examples and claims.

### SUMMARY OF THE INVENTION

In a first aspect, the invention relates to a high-pressure medical pumping device for providing discrete portions of a medical fluid in pressurized form, wherein the pumping device comprises
a fluid inlet;
a fluid outlet; and
a pumping unit fluidically connecting the fluid inlet with the fluid outlet and being adapted to generate the discrete portions of the medical fluid in pressurized form and to convey the discrete portions of the medical fluid (in a downstream direction) from the fluid inlet to the fluid outlet;
wherein the pumping unit comprises
a pump cylinder comprising a pump chamber located within the pump cylinder comprising a fluid opening, wherein the pump chamber has a variable chamber height (CH); and
a longitudinal piston comprising a first end and a second end located opposite of the first end, wherein the longitudinal piston is at least partly located within the pump chamber of the pump cylinder such that at least the first end of the piston is located within the pump chamber, and wherein the piston and the pump cylinder are arranged for back and forth stroke movement of at least one of the piston and the pump chamber relative to each other, wherein the piston is of circular shape with an outer diameter (OD),
means to exert a release force (RF) to initiate a stroke movement of the piston and/or the pump chamber to convey the fluid to the fluid outlet;
   wherein the piston comprises a fluid channel fluidically connecting the first end of the piston with the second end of the piston and being adapted to convey the medical fluid from the first or second end of the piston to the corresponding opposite end of the piston, wherein said fluid channel has a diameter corresponding to an inner diameter (ID) of the piston; and
   wherein the pumping unit is adapted to provide the discrete portions of the medical fluid in pressurized form at a pressure of at least 100 bar.

In a further aspect, the present invention provides an inhalation device for the administration of a medical liquid in aerosolized form comprising a high-pressure medical pumping device according to the invention.

In yet a further aspect, the invention provides for the use of an inhalation device comprising a high-pressure medical pumping device according to the invention to administer a medical liquid in aerosolized form to a patient in need thereof.

### BRIEF DESCRIPTION OF THE DRAWINGS

FIGS. 1A and 1B depicts a general schematic of an inhalation device comprising a pumping unit according to the present invention in default state (FIG. 1A) or primed state (FIG. 1B).

FIG. 1C shows an enlarged superposition of the two states exemplifying specific parameters of the device.

FIGS. 2A, 2B and 2C illustrate a pump cycle of a pumping unit according to the present invention.

### DETAILED DESCRIPTION OF THE INVENTION

The inventors found that specific combinations of the parameters of the pumping unit of the inhalation device allows for the creation of a compactly designed device which allows for a reasonable spraying time with a good quality of the generated aerosol.

Accordingly, in a first aspect the invention relates to a high-pressure medical pumping device for providing discrete portions of a medical fluid in pressurized form, wherein the pumping device comprises
a fluid inlet;
a fluid outlet; and
a pumping unit fluidically connecting the fluid inlet with the fluid outlet and being adapted to generate the discrete portions of the medical fluid in pressurized form and to convey the discrete portions of the medical fluid (in a downstream direction) from the fluid inlet to the fluid outlet;
wherein the pumping unit comprises
a pump cylinder comprising a pump chamber located within the pump cylinder comprising a fluid opening, wherein the pump chamber has a variable chamber height (CH); and
a longitudinal piston comprising a first end and a second end located opposite of the first end, wherein the longitudinal piston is at least partly located within the pump chamber of the pump cylinder such that at least the first end of the piston is located within the pump chamber, and wherein the piston and the pump cylinder are arranged for back and forth stroke movement of at least one of the piston and the pump chamber relative to each other, wherein the piston is of circular shape with an outer diameter (OD),
means to exert a release force (RF) to initiate a stroke movement of the piston and/or the pump chamber to convey the fluid to the fluid outlet;
   wherein the piston comprises a fluid channel fluidically connecting the first end of the piston with the second end of the piston and being adapted to convey the medical fluid from the first or second end of the piston to the corresponding opposite end of the piston, wherein said fluid channel has a diameter corresponding to an inner diameter (ID) of the piston; and
   wherein the pumping unit is adapted to provide the discrete portions of the medical fluid in pressurized form at a pressure of at least 100 bar.

In a particular embodiment, said high-pressure medical pumping device comprises a pumping unit comprising:
a pump chamber with a variable chamber height in the form of (i) a hollow cylinder (9) adapted to receive a piston in the form of a riser pipe, wherein the upstream end of said riser pipe (5) is inserted in the cylinder (9) such that the cylinder (9) is longitudinally movable on the riser pipe (5); wherein
said riser pipe (5) is having an upstream end, wherein the riser pipe (5) is
   adapted to function as a piston in the pumping unit, and
   firmly affixed to the user-facing side of a housing (1) of an inhalation device such as to be immobile relative to the housing (1); and
(ii) a lockable means in the form of a spring for storing potential energy (7) when locked and for releasing the stored energy when unlocked, the means (7) being arranged outside of, and mechanically coupled to, the cylinder (9) such that unlocking the means (7) results in a propulsive longitudinal movement of the cylinder (9) towards the downstream end of the pumping unit for providing a release force RF;
wherein the riser pipe has an outer diameter OD and an inner diameter ID;
wherein the spring has a defined spring force SF corresponding to the release force RF;
wherein the spring has a defined stroke SS which corresponds to the variable chamber height.

Said pumping unit which is also arranged within the housing is adapted to function as a piston pump, also referred to as plunger pump, wherein the riser pipe functions as the piston, or plunger, which is longitudinally moveable within the hollow cylinder. The inner segment of the hollow cylinder in which the upstream end of the riser pipe moves forms a pumping chamber which has a variable volume, depending on the position of the riser pipe relative to the cylinder.

The hollow cylinder which provides the pumping chamber is fluidically connected with the fluid reservoir, either directly or indirectly, such as by means of an optional reservoir pipe (or reservoir pipe section). Similarly, the riser pipe, whose reservoir-facing, interior (upstream) end which can be received in the hollow cylinder, is fluidically connected at its downstream or exterior end to the nozzle in a liquid-tight manner, either directly or indirectly.

In this context, the expression “hollow cylinder” refers to a part or member which is hollow in the sense that it comprises an internal void which has a cylindrical shape, or which has a segment having a cylindrical space. In other words, and as is applicable to other types of piston pumps, it is not required that the external shape of the respective part or member is cylindrical. Moreover, the expression “hollow cylinder” does not exclude an operational state of the respective part or member in which the “hollow” space may be filled with material, e.g., with a liquid to be nebulized.

The high-pressure medical pumping device for providing discrete portions of a medical fluid in pressurized form of this first aspect of the invention allows for the generation of discrete portions the medical fluid at high pressures. The term “high-pressure” as used herein in connection with either one of the pumping device, the pumping unit comprised by the pumping device or the medical fluid means, unless otherwise stated a pressure in the range of from at least about 100 bar, for example from about 100 bar to about 1000 bar or even higher, or from at least about 125 bar, for example from about 125 bar to about 750 bar or from at least about 150 bar, for example from about 150 bar to about 500 bar, or from at least about 175 bar, for example from about 175 bar to about 400 bar, or from at least about 200 bar, for example from about 200 bar to about 300 bar.

In some embodiments, the high-pressure medical pumping device according to the invention comprises a medical pumping unit which is adapted or arranged to provide the discrete portions of the medical fluid in pressurized form at a pressure of at least 150 bar, or of at least 175 bar or of at least 200 bar.

The present high-pressure medical pumping device provides discrete portions of a medical fluid in pressurized form. The term “medical fluid” as used herein is to be interpreted broadly and means a fluid or preferably a liquid that may be applied or administered to a subject, for example by inhalation, and that preferably is physiologically and/or pharmaceutically acceptable. The medical fluid is preferably non-pressurized and free of a propellant before being conveyed into the pump chamber. In some embodiments, the medical active fluid or preferably liquid to be pressurized and conveyed by the pumping unit as comprised by the present pumping device may be a water or another physiologically and/or pharmaceutically acceptable liquid compound or mixture of liquid compounds or liquid composition comprising one or more physiologically and/or pharmaceutically acceptable ingredient such as a solution or dispersion.

The medical active fluid as described above as provided in pressurized form by the high-pressure medical pumping unit of the present invention in provided in discrete portions. The terms “discrete portion” or “portion” or “unit dose” are used herein synonymously means the amount in which the medical fluid is provided by the present medical pumping device or, more specifically, the corresponding pumping unit at a single time or, in other words, upon a single actuation of the pumping device.

In some embodiments, one discrete portion of the medical fluid has a volume selected in the range of from about 1 μl to about 1000 μL or even above or from about 1 μL to about 750 μL or to about 500 μL or from about 1.5 μL to about 500 μL or to about 400 μL or from about 2 μL to about 300 μL or to about 200 μL or from about 2 μL to about 150 μL. In particular embodiments, the volume of medical fluid is selected from about 10 μl, 12 μl, 15 μl, 17 μl, 20 μl, 22 μl, 23 μl, 24 μl, 25 μl, 26 μl, 27 μl, 28 μl, and 30 μl.

The pumping device according to the present invention is suitable for the implementation in a broad variety of medical devices such as inhalation devices, and others. In some embodiments, the present pumping device is adapted to be comprised by an inhalation device for the administration of a medical fluid as described above in aerosolized form for inhalation. As the present pumping device, more specifically the present pumping unit may be provided in any size, including but not limited to miniaturized forms, in some embodiments, the present pumping device is adapted to be comprised by a mobile inhalation device, preferably by a hand-held inhalation device.

The high-pressure medical pumping device according comprises a fluid inlet as well as fluid outlet. The term “fluid inlet” as used herein is also to be interpreted broadly and generally refers to an opening or inlet through which the medical fluid to be pressurized and conveyed can enter the pumping device, for example in the form of a tubing or other fluidic connection. In some embodiments, the medical fluid may enter the present pumping device via the fluid inlet in non-pressurized form or in a form in which the fluidic pressure of the medical fluid is lower than the fluidic pressure of the medical fluid leaving the pumping device via the fluid outlet as described below after being pressurized. Accordingly, the fluid inlet of the pumping device is also sometimes referred to as “low pressure end” of the pumping unit from which the medical fluid is conveyed in a downstream direction towards the fluid outlet by the pumping unit as described below.

Correspondingly, the term “fluid outlet” as used herein generally refers to an opening or outlet to which the medical fluid in pressurized form is conveyed to and through which can exit the pumping device, for example in the form of a tubing or other fluidic connection. In some embodiments, the medical fluid may exit the present pumping device via the fluid outlet in pressurized form or in a form in which the fluidic pressure of the medical fluid is higher than the pressure of the medical fluid entering the pumping device via the fluid inlet as described above before being pressurized. Accordingly, the fluid outlet of the pumping device is also sometimes referred to as “high pressure end” of the pumping unit to which the medical fluid is conveyed in a downstream direction from the fluid inlet by the pumping unit as described below.

In some embodiments of the present high-pressure medical pumping device, the liquid inlet is fluidically connected or connectable to a reservoir for holding the medical fluid. The term “reservoir” as used herein may be interpreted broadly as relating to container which is suitable for holding or storing the medical fluid to be pressurized and dispensed. In some embodiments, the reservoir may be in the form of a dimensionally stable or flexible container, for example a dimensionally stable or flexible bottle with an outlet opening which may be fluidically connected to the fluid inlet of the pumping device. In some embodiments, the reservoir is adapted to hold the medical fluid in non-pressurized form or, in other words, at atmospheric pressure. In further embodiments, the reservoir may be firmly attached to or connected with the fluid inlet of the pumping device. In alternative embodiments, however, the reservoir may be designed to be removably attached to the fluid inlet of the present inhalation device.

In some embodiments, the reservoir may be designed to be collapsible, such as by means of a flexible or elastic wall. The effect of such design is that upon repeated use of the device which involves progressive emptying of the reservoir, the flexible or elastic wall buckles or folds such as to reduce the internal volume of the reservoir, so that the negative pressure which is necessary for extraction of a certain amount of liquid is not required to increase substantially over the period of use. In particular, the reservoir may be designed as a collapsible bag. The advantage of a collapsible bag is that the pressure inside the reservoir is almost independent of the filling level, and the influence of thermal expansion is almost negligible. Also, the construction of such a reservoir type is rather simple and already well established. A similar effect can be achieved with a rigid or dimensionally stable container which has a moveable bottom (or wall) by means of which the interior volume of the reservoir can also be successively reduced.

In some embodiments of the present pumping device, the fluid outlet is fluidically connected to a nozzle for discharging the pressurized and conveyed discrete portions of the medical fluid in pressurized form. In embodiments as described above in which the present high-pressure medical pumping device is comprised by an inhalation device, such a nozzle may be adapted to aerosolize or, in other words, nebulize the pressurized discrete portions of medical fluid. Suitable nozzles in this regard comprise but are not limited to, for example, swirl nozzles, impingement-type nozzles, flat fan nozzles, hollow cone nozzles, full cone nozzles, solid stream nozzles, and Raleigh nozzles (nozzle plates). In more specific embodiments, the high-pressure pumping unit may be connected with an impingement-type nozzle for aerosolizing the medical fluid by collision of at least two liquid jets of the medical fluid. Such nozzles, especially such impingement-type nozzles require a considerable fluidic pressure of the liquid medium to be aerosolized and, accordingly, are especially suited to be combined with the present high-pressure medical pumping device.

The present high-pressure medical pumping device further comprises a pumping unit fluidically connecting the fluid inlet with the fluid outlet and being adapted to generate the discrete portions of the medical fluid in pressurized form and to convey the discrete portions of the medically active fluid (in a downstream direction) from the fluid inlet to the fluid outlet.

The term “fluidically connecting” as used herein is to be interpreted broadly and refers to all structures through which a fluid or liquid, specifically the medical fluid, may be transported from one part of the device to another part of the device, preferably without significant loss of the liquid to be transported. Such a fluidic connection may be implemented, for example, by a tubing through which the fluid may be transported or conveyed or by another fluidic structure that allows for the lossless transport of such fluid as, for example by the pumping unit of the present high-pressure pumping device.

In some embodiments, the pumping unit is adapted to function as a piston pump, also referred to as plunger pump comprising a piston or plunger, which is longitudinally moveable within a hollow cylinder. The inner segment of the hollow cylinder in which at least a segment of the piston moves forms a pumping chamber which has a variable chamber height (CH), depending on the position of the piston relative to the cylinder. Said variable chamber height may be achieved by movement of the hollow cylinder or by movement of the longitudinal piston.

The term “variable chamber height” refers to the variation in height and does not necessarily include the total height of the pump chamber. The chamber may have a minimal height plus the variable chamber height. As such the maximal height of the pump chamber corresponds to the minimal chamber height plus the maximal variable chamber height.

In some embodiments of the invention, the means to exert a release force are a lockable means in the form of a spring for storing potential energy. Said spring has a defined spring force (SF), which corresponds to the release force and a defined spring stroke (SS).

In the context of the present invention, the term “spring force” refers to the force that the spring is able to exert on the hollow cylinder and the pump chamber upon release.

The term “stroke of the spring” refers to the length of compression during the use of the pumping unit. The variable chamber height (CH) corresponds to said spring stroke (SS).

The maximum of the variable chamber height (CH) during operation is preferably between 6 and 12 mm. In some embodiments the variable chamber height is between 8 and 10 mm. In some embodiments the variable chamber height is selected from 6, 7, 8, 9, 10, 11 and 12 mm. In a preferred embodiment the variable chamber height is 9 mm.

In further embodiments, the present high-pressure medical pumping device may comprise a check valve fluidically connecting the fluid inlet with the pumping unit (and being located between the fluid inlet and the pumping unit).

Accordingly, the pumping unit of the present high-pressure pumping device comprises a pump cylinder comprising a pump chamber located within the pump cylinder, the pump chamber comprising a fluid opening. As described above, the pump cylinder may also be referred to as a “hollow cylinder” corresponding to a part or member which is hollow in the sense that it comprises an internal void which has a cylindrical shape, or which has a segment having a cylindrical space. In other words, and as is applicable to other types of piston pumps, it is not required that the external shape of the respective part or member is cylindrical. Moreover, the expression “hollow cylinder” does not exclude an operational state of the respective part or member in which the “hollow” space may be filled with material, e.g. with a liquid to be nebulized.

The pump chamber further comprises a fluid opening in addition to the opening of the pump cylinder into which in the operational state the longitudinal piston is introduced. The fluid opening may be, depending of the design of the pumping unit as described further below, fluidically connected with either the fluid inlet or the fluid outlet of the pumping device and, accordingly, may either be located at the low-pressure side of the pumping unit to allow the medical fluid to be conveyed and pressurized to enter the pump chamber, or may be located at the high-pressure side of the pumping unit to allow the pressurized discrete portions of medical fluid to exit the pump chamber. In the first case the fluid opening may be in fluidic connection with the fluid inlet of the pumping device and in the second case the fluid opening may be in fluidic connection with the fluid outlet of the pumping device.

The present high-pressure medical pumping device further comprises a longitudinal piston comprising a first end and a second end located opposite of the first end, wherein the longitudinal piston is at least partly located within the pump chamber of the pump cylinder such that at least the first end of the piston is located within the pump chamber. The longitudinal piston, in some embodiments, is a longitudinal cylinder with preferably a circular cross-sectional area with a first end to be introduced into the internal void of the pump cylinder. In some embodiments, the first and the second end of the longitudinal piston may be located within the pump chamber (in the operational state of the pumping unit) which means that the entire longitudinal piston is located within the cylindrical internal void (pump chamber) of the piston. In alternative embodiments, the longitudinal piston may be located within the pump chamber (in the operational state) such that only the first end is located within the pump chamber and, accordingly, the second end is located outside of the pump chamber (in the operational state).

In some embodiments, the piston is in form of a riser pipe, which is adapted to function as a piston in the pumping unit.

The volume of the pump chamber is preferably a result of the outer diameter (OD) of the circular piston and the variable chamber height.

The piston and the pump cylinder of the pumping unit are arranged for back-and-forth stroke movement of at least one of the piston and the pump chamber relative to each other. During such back-and-forth stroke movement of either the piston relative to the pump chamber or of the pump chamber relative to the piston or of both, the piston and the pump chamber relative to the respective other one, the volume of the pump chamber as defined by the corresponding (internal surface) of the pump chamber and (the outer surface of) the first end of the piston varies according to the corresponding position of the piston within pump chamber during the longitudinal back-and-forth stroke movement. This allows for the compression and conveyance of the medical fluid when introduced into the pump chamber during a stroke-like movement of either the longitudinal piston or the pump cylinder or of both relative to each other in which the volume of the pump chamber is reduced.

In some embodiments, one pumping cycle of the umping unit comprised by the present high-pressure medical pumping device starts with situation in which the longitudinal piston is positioned maximally within the pump cylinder (resulting in a minimal volume of the pump chamber) and comprises a relative movement of the piston to a position in which it is maximally extracted from the pump cylinder (repulsive movement; resulting in a maximal volume of the pump chamber) followed by a relative back movement of the piston to its initial position (propulsive movement). In some embodiments, one discrete portion of the medical fluid in pressurized form to be delivered by the present high-pressure medical pumping device is delivered per pumping cycle.

As described above, the volume of the pump chamber may vary in the course of the stroke-like movement of either one of the piston or the pump cylinder. In some embodiments, the pump chamber (as defined by the inner walls of the pump cylinder and the first end of the piston) of the present high-pressure medical pumping device has a volume selected within the range of from about 1 μL to about 100 μL, preferably of from about 5 to 50 μl, more preferably 15 μL to about 30 μL. For the avoidance of doubt, this volume refers to the maximum volume of the pump chamber in an operational state of the pumping unit in which the longitudinal piston is in its outermost position relative to the pump cylinder.

The volume of fluid (e.g., of medically active liquid) that is pumped by the pumping unit in one pumping cycle is preferably in the range from about 2 to about 150 μl. In particular, the volume may range from about 5 to about 50 μl, or from about 10 to about 30 μl, respectively. This volume ranges are nearly the same as the volume of liquid phase that is contained in one unit of aerosol generated by the inhalation device, perhaps with minor differences due to minute losses of liquid in the device. In particular embodiments, the volume of fluid is selected from about 10 μl, 12 μl, 15 μl, 17 μl, 20 μl, 22 μl, 23 μl, 24 μl, 25 μl, 26 μl, 27 μl, 28 μl, and 30 μl.

In some embodiments, the outer diameter of the longitudinal piston (as measured perpendicular to the longitudinal main axis of the piston) is essentially constant over and preferably corresponds or essentially corresponds the inner diameter of the pump chamber over the entire segment of the longitudinal piston introduced into the pump chamber.

In further embodiments, the longitudinal piston as comprised by the present high-pressure medical pumping device may have an outer diameter (OD, perpendicular to the main axis) selected within a range of from about 1.6 mm to about 2.2 mm, between 1.7 and 2.0 mm. In some embodiments, the outer diameter of the longitudinal piston selected from 1.6 mm, 1.7 mm, 1.8 mm, 1.9 mm, 2.0 mm, 2.1 mm and 2.2. In some embodiments the outer diameter of the longitudinal piston is 1.8 mm.

The longitudinal piston of the pumping unit comprises a fluid channel fluidically connecting the first end of the piston with the second end of the piston and being adapted to convey the medical fluid from the first or second end of the piston to the corresponding opposite end of the piston. This fluid channel or, more specifically, the second end of this fluid channel, may be, depending of the design of the pumping unit as described further below, fluidically connected with either the fluid inlet or the fluid outlet of the pumping device and, accordingly, may either be located at the low-pressure side of the pumping unit to allow the medical fluid to be conveyed and pressurized to enter the pump chamber, or may be located at the high-pressure side of the pumping unit to allow the pressurized discrete portions of medical fluid to exit the pump chamber. In the first case the (second end of) the fluid channel may be in fluidic connection with the fluid inlet of the pumping device and in the second case the (second end of) the fluid channel may be in fluidic connection with the fluid outlet of the pumping device.

In some embodiments, the fluid channel may extend along the entire length of the longitudinal piston from the first end of the piston to the opposite second end the piston. In further embodiments, the fluid channel may have a circular or non-circular cross-sectional shape, preferably, a circular cross-sectional shape. In yet further embodiments, the fluid channel may have a main axis extending from the first end of the piston to the second end of the piston, which, in some embodiment, corresponds to the main axis of the longitudinal piston. In further embodiments, the fluid channel may have a main axis connecting the centers of the first and second end of the piston and the fluid channel may have a diameter corresponding to an inner diameter of the piston perpendicular to the main axis selected within the range of from about 0.1 mm to about 1.0 mm, preferably of from about 0.3 mm to about 0.7 mm. In some embodiments the inner diameter is selected from 0.3 mm, 0.4 mm, 0.5 mm, 0.6 mm, and 0.7 mm. In preferred embodiments the inner diameter of the longitudinal piston and the fluid channel is 0.5 mm.

The inventors found that specific ratios of the inner diameter of to the outer diameter of the longitudinal piston allow for improved spraying behavior. It is therefore preferred in some embodiments of the invention that the ratio of the outer diameter in mm to the inner diameter in mm is greater than 3 to 1. In particular embodiments, the ratio of the outer diameter in mm to the inner diameter of the longitudinal piston in mm is between 3.2 to 1 and 4 to 1. In some embodiments the ratio is between 3.4 to 1 and 3.8 to 1. In some embodiments, the ratio of the outer diameter of the longitudinal piston in mm to the inner diameter of the piston in mm is selected from 3.2 to 1, 3.3 to 1, 3.4 to 1, 3.5 to 1, 3.6 to 1, 3.7 to 1, 3.8 to 1 and 3.9 to one. In preferred embodiments, the ratio of the outer diameter of the longitudinal piston in mm to the inner diameter of the longitudinal piston in mm is 3.6 to 1.

The length of the longitudinal piston is preferably selected from between 10 and 30 mm. In some embodiments the length is between 15 and 25 mm. In some embodiments the length of the longitudinal piston is selected from 15.0 mm, 15.5 mm, 16.0 mm, 16.5 mm, 17.0 mm, 17.5 mm, 18.0 mm, 18.5 mm, 19.0 mm, 19.5 mm, and 20.0 mm. In a preferred embodiment the length of the longitudinal piston is 17.5 mm.

In some embodiments of the present high-pressure medical pumping device, the fluid opening of the pump cylinder may be fluidically connected to the fluid inlet of the pumping device (and optionally to the reservoir) and located downstream of the fluid inlet of the pumping device and the second end of the piston may be fluidically connected to the fluid outlet of the pumping device. In further embodiments, the pumping unit may comprise a check valve and the check valve may be comprised by the pump cylinder and/or may be positioned adjacent to the fluid inlet of the pump cylinder. In these embodiments, the longitudinal piston may be arranged in a fixed position within the pumping unit such that the piston is immobile during back-and-forth stroke movement of the pump chamber.

In alternative embodiments of the present high-pressure medical pumping device, the fluid opening of the pump cylinder may be fluidically connected to the fluid outlet of the pumping device and located upstream of the fluid outlet of the pumping device and the second end of the piston may be connected to the fluid inlet of the pumping device. In further embodiments, the pumping unit may comprise a check valve comprised by the piston, and optionally positioned at the first end of the piston. In these embodiments, the pump chamber may be arranged in a fixed position within the pumping unit such that the pump chamber is immobile during back-and-forth stroke movement of the piston.

In some embodiments of the present high-pressure medical pumping device, one discrete portion of the medical fluid in pressurized form is generated by one stroke movement of at least one of the piston and the pump cylinder.

The pumping unit of the present high-pressure medical pumping device is adapted to provide the discrete portions of the medical fluid in pressurized form at a pressure of at least 100 bar (10 MPa), specifically at a pressure of in the range of from at least about 100 bar or of at least 150 bar or of at least 175 or of at least 200 bar, for example from about 100 bar to about 1000 bar or even higher, or from at least about 125 bar, for example from about 125 bar to about 750 bar or from at least about 150 bar, for example from about 150 bar to about 500 bar, or from at least about 175 bar, for example from about 175 bar to about 400 bar, or from at least about 200 bar, for example from about 200 bar to about 300 bar.

The high-pressure medical pumping device comprises means to exert a release force to initiate a stroke movement of the piston and/or the pump chamber

In some embodiments, the present high-pressure medical pumping device or, more specifically, the pumping unit comprises a drive unit adapted for driving the stroke movement of at least one of the piston and the pump cylinder as said means to exert a release force. The drive unit allows for the propulsion or retraction of at least one of the longitudinal piston and the pump cylinder relative to each other, especially during the part of the pumping cycle in which the volume of the pump chamber is reduced and, accordingly, the discrete portion of the medical fluid is to be compressed. The drive unit may be in any suitable form that allows for the generation of a stroke-like back-and-forth movement of the piston relative to the pump cylinder. In some embodiments, it may comprise a motor, such as an electrical motor or a means for the storage and release of potential energy to be transform into kinetic energy of the piston and/or the cylinder, for example a spring, a compressed gas or a compressed elastomeric material.

In particular embodiments, the drive unit may be adapted to exert a release force on at least one of the piston and pump cylinder of at least 30 N or at least 35 N or at least 40 N or at least 50 N to about 200 N or to about 150 N or to about 100 N. in further particular embodiments, the drive unit may be adapted to exert a driving force on at least one of the piston and pump cylinder in the range of from about 30 N to about 150 N or to about 100N, or selected within the range of from about 35 N to about 80 N.

In further embodiments, the pumping unit of the present high-pressure medical pumping device may comprise a lockable means for storing potential energy when locked and for releasing the stored energy when unlocked, the means being arranged outside of and being mechanically coupled to at least one of the pump cylinder and the piston such that unlocking the means results in a propulsive longitudinal movement of at least one of the pump cylinder and the piston towards the corresponding other one of the pump cylinder and the piston. In some embodiments, the means for storing potential energy as referred to above may comprise a spring, such as a spring made of steel or another elastic metal or material. In particular embodiments, such as a spring may have a load within the ranges described above in connection with driving force of the drive unit of at least about 30 N or at least 35 N or at least 40 N or at least 50 N to about 200 N or to about 150 N or to about 100N in a deflected state or selected in the range of from about 30 N to about 150 N or to about 100N, or selected within the range of from about 35 N to about 80 N in a deflected state. In further embodiments, the pumping unit may further comprise a lock for locking the lockable means. This may allow the driving means to be tensioned or loaded and, in a separate step, to be triggered by unlocking the lockable means for storing potential energy to drive the pumping unit.

In some embodiments of the invention, the means for exerting a release force comprise a spring.

The inventors found that a specific ratio of the release force in N to the maximum variable chamber height of the pump chamber in mm can improve aerosol quality and/or spray duration.

The ratio of the release force in N to the maximum variable chamber height in mm is preferably greater than 5.0. In some embodiments the ratio of the release force in N to maximum variable chamber height in mm is between 5.5 and 8.0 to 1. In some embodiments the ratio is between 6.0 and 70 to 1. In particular embodiments of the invention the ratio of the release force in N to the maximum variable chamber height in mm is selected from 5.5, 5.8, 6.0, 6.2, 6.3, 6.4, 6.5, 6.6, 6.7, 6.8, 6.9, 7.0, 7.2, and 7.5 to 1. In preferred embodiments the ratio of the release force in N to the maximum variable chamber height in mm is 6.6 to 6.7 to 1.

For some embodiments it is preferable to associate a specific release force with a defined outer diameter of the longitudinal piston. As such in preferred embodiments of the invention the ratio of the release force in N to the outer diameter of the longitudinal piston in mm is greater than 30 to 1. Preferably, the ratio of the release force in N to the outer diameter of the longitudinal piston in mm is between 30.5 and 36.0 to 1. In some embodiments the ratio of the release force in N to the outer diameter of the longitudinal piston in mm is between 32.0 and 34.0 to 1. In some embodiments of the invention, the ratio of the release force in N to the outer diameter of the longitudinal piston in mm is between 33.0 and 33.6 to 1. In some embodiments the ratio of the release force in N to the outer diameter of the longitudinal piston in mm is selected from 30.5, 31.0, 31.5, 32.0, 32.3, 32.5, 32.7, 33.0, 33.2, 33.3, 33.4, 33.5, 33.7, 34.0, 34.3, 34.5, 34.7, 35.0 to 1. In particular preferred embodiments the release force in N to the outer diameter of the longitudinal piston in mm is 33.3 to 1.

The inventors found that the ratios as defined above may be combined. As such, in a preferred embodiment of the invention the pump chamber comprises a spring and is adapted to receive a longitudinal piston as defined above, wherein the ratio of the outer diameter of the longitudinal piston to the release force is as defined above and the ratio of the release force to the variable chamber height is as defined above.

In particular embodiments, the invention relates to a pump chamber as defined above or an inhalation device comprising a pump chamber as defined above, comprising a lockable means for storing potential energy in the form of a spring and the pump chamber is adapted to receive a longitudinal piston, wherein embodiments the ratio of the release force in N to the outer diameter of the longitudinal piston in mm is selected from 30.5, 31.0, 31.5, 32.0, 32.3, 32.5, 32.7, 33.0, 33.2, 33.3, 33.4, 33.5, 33.7, 34.0, 34.3, 34.5, 34.7, 35.0 to 1, preferably 33.3 to 1 and the ratio of the release force in N to the variable chamber height in mm is selected from 5.5, 5.8, 6.0, 6.2, 6.3, 6.4, 6.5, 6.6, 6.7, 6.8, 6.9, 7.0, 7.2, and 7.5 to 1, preferably 6.6 or 6.7 to 1.

In a particular embodiment, the invention relates to an inhalation device as defined above, wherein
the ratio of the release force in N to the variable chamber height in mm is 6.6 to 6.7 to 1;
the ratio of the outer diameter of the longitudinal piston in mm to the inner diameter of the longitudinal piston in mm is 3.6 to 1; and
the ratio of the release force in N to the outer diameter of the longitudinal piston in mm is 33.3.

The pumping unit according to the present invention allows for an optimized spray time with higher volumes, while maintaining a compact size of the devices. In particular the spray time of a device according to the invention is lower than 3 seconds. With longer spray times patient compliance and the effectiveness of the inhalation can be reduced.

In a second aspect, the present invention provides for an inhalation device for the administration of a medical fluid in aerosolized form comprising a high-pressure medical pumping device according to the first aspect of the invention as described in detail above. As already mentioned, in some embodiments, the inhalation device according to this aspect of the invention may be a mobile inhalation device, more particularly a hand-held inhalation device that may be carried and used a user by holding and operating it with one or both hands of such user. Such inhalation device are, for example, disclosed in documents WO 91/14468 A1 or WO 2018/197730 A1 as already referred to above.

For the avoidance of doubt, it should be noted that all embodiments, features, definitions, explanations and combinations thereof as disclosed in connection with the pumping device of the first aspect of the invention also apply to this second aspect of the invention as well as all further aspects of the invention although not being repeated in connection with the present and all further aspects of the invention.

In a third aspect, the present invention provides for a piston for the pumping unit of a high-pressure medical pumping device according to the first aspect of the invention. The piston according to this third aspect of the invention comprises an injection-moldable polymeric material as disclosed in detail above in connection with the first aspect of the invention. The piston according to this aspect of the invention is adapted to be comprised by the medical high-pressure pumping device according to the first aspect of the invention, more specifically adapted to be comprised by the pumping unit of the medical high-pressure pumping device according to the first aspect of the invention.

The parts of the pumping medical pumping device of the present invention may be made of any suitable material. In particular, the pump cylinder is made of a preferably form-stable material.

Said form-stable material may preferably be a polymeric material or a metal. The hollow cylinder is preferably made of a polymeric material. In preferred embodiments, the pump cylinder is made of a polymeric material comprising a polymer selected from the group comprising polyethylene, poly lactic acid, poly propylene and PEEK.

The high-pressure medical pumping device according to any one of the preceding claims, wherein the pump cylinder is made of a polymeric material.

In some embodiments of the invention, the pump cylinder of the high-pressure medical umping device is made of a thermoplastic polymeric material. In preferred embodiments of the invention, the pump cylinder is made of a polymeric material selected from the group consisting of acrylonitrile butadiene styrene (ABS), polyamides, polylactic acid (polylactide, PLA), polybenzimidazole PBI), polyether sulfone (PES), polyoxymethylene (POM), polyether ether ketone (PEEK), polyphenylene oxide (PPO).

In a particular embodiment the invention relates to a high-pressure medical pumping device as defined above, wherein the pump cylinder is made of a s polyether ether ketone (PEEK).

The invention further relates to the following numbered embodiments.
1. A high-pressure medical pumping device for providing discrete portions of a medical fluid in pressurized form, wherein the pumping device comprises
   a fluid inlet;
   a fluid outlet; and
   a pumping unit fluidically connecting the fluid inlet with the fluid outlet and being adapted to generate the discrete portions of the medical fluid in pressurized form and to convey the discrete portions of the medical fluid (in a downstream direction) from the fluid inlet to the fluid outlet;
   wherein the pumping unit comprises
   a pump cylinder comprising a pump chamber located within the pump cylinder comprising a fluid opening, wherein the pump chamber has a variable chamber height (CH); and
   a longitudinal piston comprising a first end and a second end located opposite of the first end, wherein the longitudinal piston is at least partly located within the pump chamber of the pump cylinder such that at least the first end of the piston is located within the pump chamber, and wherein the piston and the pump cylinder are arranged for back and forth stroke movement of at least one of the piston and the pump chamber relative to each other, wherein the piston is of circular shape with an outer diameter (OD),
   means to exert a release force (RF) to initiate a stroke movement of the piston and/or the pump chamber to convey the fluid to the fluid outlet;
   wherein the piston comprises a fluid channel fluidically connecting the first end of the piston with the second end of the piston and being adapted to convey the medical fluid from the first or second end of the piston to the corresponding opposite end of the piston, wherein said fluid channel has a diameter corresponding to an inner diameter (ID) of the piston; and
   wherein the pumping unit is adapted to provide the discrete portions of the medical fluid in pressurized form at a pressure of at least 100 bar.
2. The high-pressure medical pumping device according to embodiment 1, wherein the ratio of outer diameter to inner diameter of the longitudinal piston (OD/ID) is at least 3.2 to 1; and/or
   the ratio of release force in N to variable chamber height in mm (RF/CH) is at least 5.0 to 1; and/or
   the ratio of release force in N to outer diameter in mm (RF/OD) is at least 30.5 to 1.
3. The high-pressure medical pumping device according to embodiment 1 or 2, wherein the outer diameter of the piston is between 1.7 and 2.5 mm.
4. The high-pressure medical pumping device according to any of the preceding embodiments, wherein the inner diameter of the piston is 0.5 mm.
5. The high-pressure medical pumping device according to any of the preceding embodiments, wherein the release force is between 50 and 70 N.
6. The high-pressure medical pumping device according to any of the preceding embodiments, wherein the variable chamber height is between 6 and 12 mm.
7. The high-pressure medical pumping device according to any of the preceding embodiments, wherein the medical pumping unit is adapted to provide the discrete portions of the medical fluid in pressurized form at a pressure of at least 150 bar, or of at least 175 bar or of at least 200 bar.
8. The high-pressure medical pumping device according to any of the preceding embodiments, wherein the medical pumping device comprises a check valve fluidically connecting the fluid inlet with the pumping unit (and being located between the fluid inlet and the pumping unit).
9. The high-pressure medical pumping device according to any one of the preceding embodiments, wherein the pumping device is adapted to be comprised by an inhalation device, preferably by a hand-held inhalation device.
10. The high-pressure medical pumping device according to any one of the preceding embodiments, wherein the pumping device is adapted to be comprised by an inhalation device for the administration of the medical fluid in aerosolized form for inhalation.
11. The high-pressure medical pumping device according to any one of the preceding embodiments, wherein the liquid inlet is fluidically connected to a reservoir for holding the medical fluid, for example a reservoir in the form of a dimensionally stable or flexible container.
12. The high-pressure medical pumping device according to any one of the preceding embodiments, wherein the fluid outlet is fluidically connected to a nozzle for discharging and optionally aerosolizing the conveyed medical fluid in pressurized form, for example an impingement-type nozzle for aerosolizing the medical fluid by collision of at least two liquid jets of the medical fluid.
13. The high-pressure medical pumping device according to any one of the preceding embodiments, wherein the pump cylinder comprises a fluid inlet fluidically connected to fluid inlet of the pumping device (and optionally to the reservoir) and located downstream of the fluid inlet of the pumping device and wherein the second end of the piston is fluidically connected to the fluid outlet of the pumping device.
14. The high-pressure medical pumping device according to embodiment 13, wherein the pumping unit comprises a check valve and wherein the check valve is comprised by the pump cylinder and/or is positioned adjacent to the fluid inlet of the pump cylinder.
15. The high-pressure medical pumping device according to embodiment 13 or 14, wherein the piston is arranged in a fixed position within the pumping unit such that the piston is immobile during back-and-forth stroke movement of the pump chamber.
16. The high-pressure medical pumping device according to any one of embodiments 1 to 13, wherein the pump cylinder comprises a fluid outlet fluidically connected to the fluid outlet of the pumping device and located upstream of the fluid outlet of the pumping device and wherein the second end of the piston is connected to the fluid inlet of the pumping device.
17. The high-pressure medical pumping device according to embodiment 16, wherein the pumping unit comprises a check valve comprised by the piston, and optionally positioned at the first end of the piston.
18. The high-pressure medical pumping device according to embodiment 16 or 17, wherein the pump chamber is arranged in a fixed position within the pumping unit such that the pump chamber is immobile during back-and-forth stroke movement of the piston.
19. The high-pressure medical pumping device according to any one of the preceding embodiments, wherein one discrete portion of the medical fluid in pressurized form is generated by one stroke move of at least one of the piston and the pump cylinder.
20. The high-pressure medical pumping device according to any one of the preceding embodiments, wherein the pump cylinder is made of a polymeric material.
21. The high-pressure medical pumping device according to any one of the preceding embodiments, wherein the pump cylinder is made of a thermoplastic polymeric material.
22. The high-pressure medical pumping device according to any one of the preceding embodiments, wherein the pump cylinder is made of a polymeric material selected from the group consisting of polyacrylic acids, (such as poly(methyl methacrylate (PMMA)), acrylonitrile butadiene styrene (ABS), polyamides, polylactic acid (polylactide, PLA), polybenzimidazole PBI), polyether sulfone (PES), polyoxymethylene (POM), polyether ether ketone (PEEK), polyetherimide (PEI), polyethylene (PE, such as ultra-high-molecular-weight polyethylene (UHMWPE), high-density polyethylene (HDPE), medium-density polyethylene), low-density polyethylene (LDPE)), polyphenylene oxide (PPO), polyphenylene sulphide (PPS), polypropylene (PP), polystyrene (PS), polyvinyl chloride (PVC), polyvinylidene fluoride PVDF) and polytetrafluorethylene (PTFE).
23. The high-pressure medical pumping device according to any one of the preceding embodiments, wherein pump cylinder is made of a material selected from the group consisting of polyacrylic acids, (such as poly(methyl methacrylate (PMMA)), acrylonitrile butadiene styrene (ABS), polyamides, polylactic acid (polylactide, PLA), polybenzimidazole PBI), polyether sulfone (PES), polyoxymethylene (POM), polyether ether ketone (PEEK), polyetherimide (PEI), polyethylene (PE, such as ultra-high-molecular-weight polyethylene (UHMWPE), high-density polyethylene (HDPE), medium-density polyethylene), low-density polyethylene (LDPE)), polyphenylene oxide (PPO), polyphenylene sulphide (PPS), polypropylene (PP), polystyrene (PS), polyvinyl chloride (PVC), polyvinylidene fluoride PVDF) and polytetrafluorethylene (PTFE).
24. The high-pressure medical pumping device according to any one of the preceding embodiments, wherein the pump cylinder is made of a s polyether ether ketone (PEEK).
25. The high-pressure medical pumping device according to any one of the preceding embodiments, wherein the means to exert a release force on the pump chamber comprises a lockable means for storing potential energy when locked and for releasing the stored energy when unlocked, the means being arranged outside of and being mechanically coupled to at least one of the pump cylinder and the piston such that unlocking the means results in a propulsive longitudinal movement of at least one of the pump cylinder and the piston towards the corresponding other one of the pump cylinder and the piston.
26. The high-pressure medical pumping device according to embodiment 25, wherein the means for storing potential energy comprises a spring, such as a spring having a load of at least 50 N in a deflected state.
27. The high-pressure medical pumping device according to embodiment 25 or 26, wherein the pumping unit further comprises a lock for locking the lockable means.
28. The high-pressure medical pumping device according to any one of the preceding embodiments, wherein one discrete portion of the medical fluid is delivered per pumping cycle.
29. The high-pressure medical pumping unit according to any one of the preceding embodiments, wherein one portion of the medical fluid has a volume selected in the range of from 2 μL to about 150 μL.
30. An inhalation device for the administration of a medical liquid in aerosolized form comprising a high-pressure medical pumping device according to any one of embodiments 1 to 29.

In a particular aspect, the invention relates to the following specific numbered embodiments:

A1. A pumping unit for a hand-held inhalation device, the pumping unit having:
an upstream end that is fluidically connectable to a reservoir;
a downstream end that is fluidically connectable to a nozzle;
the pumping unit comprising:
a pump chamber with a variable chamber height in the form of (i) a hollow cylinder (9) adapted to receive a piston in the form of a riser pipe, wherein the upstream end of said riser pipe (5) is inserted in the cylinder (9) such that the cylinder (9) is longitudinally movable on the riser pipe (5); wherein
said riser pipe (5) is having an upstream end, wherein the riser pipe (5) is
   adapted to function as a piston in the pumping unit, and
   firmly affixed to the user-facing side of a housing (1) of an inhalation device such as to be immobile relative to the housing (1); and
(ii) a lockable means in the form of a spring for storing potential energy (7) when locked and for releasing the stored energy when unlocked, the means (7) being arranged outside of, and mechanically coupled to, the cylinder (9) such that unlocking the means (7) results in a propulsive longitudinal movement of the cylinder (9) towards the downstream end of the pumping unit for providing a release force RF;
wherein the riser pipe has an outer diameter OD and an inner diameter ID;
wherein the spring has a defined spring force SF corresponding to the release force RF;
wherein the spring has a defined stroke SS which corresponds to the variable chamber height.

A2. The pumping unit according to embodiment A1, wherein the ratio of outer diameter to inner diameter of the riser pipe (OD/ID) is at least 3.2 to 1; and/or
the ratio of spring force in N to stroke of the spring in mm (SF/SS) is at least 5.0 to 1; and/or the ratio of spring force in N to outer diameter in mm (SF/OD) is at least 30.5 to 1.

A3. The pumping unit according to embodiment A1 or A2, wherein the outer diameter of the riser pipe is between 1.7 and 2.5 mm.

A4. The pumping unit according to any of the preceding embodiments, wherein the inner diameter of the riser pipe is 0.5 mm.

A5. The pumping unit according to any of the preceding embodiments, wherein the spring force is between 50 and 70 N.

A6. The pumping unit according to any of the preceding embodiments, wherein the stroke of the spring is between 6 and 12 mm.

A7. The pumping unit for a handheld inhalation device according to any of the preceding embodiments, wherein the pumping unit is a high-pressure pumping unit adapted to expel fluid (F) at a pressure of at least 50 bar.

A8. The pumping unit for a handheld inhalation device according to any of the preceding embodiments, wherein the pumping unit further comprises an inlet valve positioned in the hollow cylinder.

A9. The pumping unit according to embodiment 8, wherein the inlet valve is adapted to open only when the pressure difference between an upstream and a downstream side of the inlet valve is above a predefined threshold value, and wherein the predefined threshold value is in the range from 1 to 20 mbar.

A10. The pumping unit according to any one of the preceding embodiments, wherein the ratio of outer diameter to inner diameter of the riser pipe (OD/ID) is at least 3.2 to 1.

A11. The pumping unit according to any one of the preceding embodiments, wherein the ratio of spring force in N to stroke of the spring in mm (SF/SS) is at least 5.0 to 1.

A12. The pumping unit according to any one of the preceding embodiments, wherein the ratio of outer diameter to inner diameter of the riser pipe (OD/ID) is at least 3.2 to 1 and wherein the ratio of spring force in N to stroke of the spring in mm (SF/SS) is at least 5.0 to 1.

A13. The pumping unit according to any one of the preceding embodiments, wherein the hollow cylinder is made of a form-stable material.

A14. The pumping unit according to embodiment A13, wherein the hollow cylinder is made of metal or a polymeric material.

A15. The pumping unit according to embodiment A13 or A14, wherein the hollow cylinder is made of a polymeric material comprising a polymer selected from the group comprising polyethylene, poly lactic acid, poly propylene and PEEK.

A16. Use of a pumping unit according to any one of the preceding embodiments for the nebulization of an aerosol in a portable handheld inhalation device.

A17. A hand-held inhalation device for delivering a nebulized medically active aerosol for inhalation therapy, comprising
(a) a housing (1) having a user-facing side;
(b) an impingement-type nozzle (6) for generating the nebulized aerosol by collision of at least two liquid jets, the nozzle (6) being firmly affixed to the user-facing side of the housing (1) such as to be immobile relative to the housing (1);
(c) a fluid reservoir (2) arranged within the housing (1);
(d) a riser pipe having an upstream end, wherein the riser pipe (5) is
   adapted to function as a piston in the pumping unit, and
   firmly affixed to the user-facing side of a housing (1) of an inhalation device such as to be immobile relative to the housing (1);
(e) a pumping unit according to any one of embodiments 1 to 15.

### DETAILED DESCRIPTION OF THE DRAWINGS

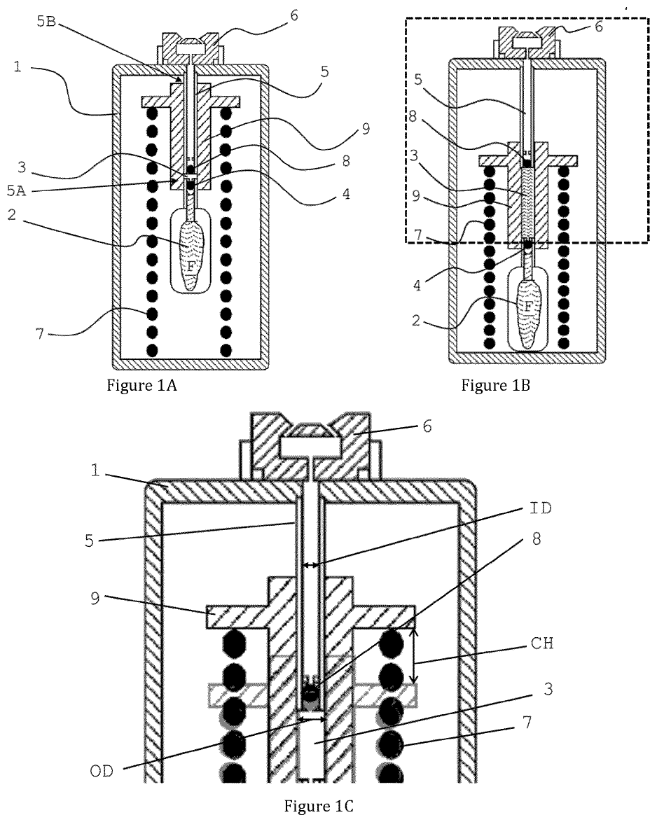

In FIG. 1A, one of the embodiments of an inhalation device comprising a high-pressure medical pumping device according to the invention is depicted schematically and not-to-scale. FIG. 1A shows the situation prior to first use.

The inhalation device comprises a housing (1), which is preferably shaped and dimensioned such that it can be held with one hand and can be operated by one finger, e.g. a thumb or index finger (not shown). A fluid reservoir (2) for the storage of a medically active liquid (F) is located inside the housing (1). The depicted reservoir (2) is designed to be collapsible so that in the course of the emptying of the reservoir by the repeated use of the device, the soft or elastic walls deform such that the negative pressure required for withdrawing liquid from the reservoir remains substantially constant over time. A similar effect could be achieved with a rigid container that has a movable bottom by means of which the interior volume of the reservoir can also successively be reduced (not shown).

Furthermore, the inhalation device comprises a pumping unit with a pump cylinder (9) within the housing (1) which forms a pumping chamber (3) for the generation of the desired pressure which is necessary for emitting liquid (F) and nebulizing the same. The pumping unit may also comprise further components not depicted in the drawing, such as a push button, locking device, etc.

As a means for exerting a release force (7), a spring is provided which is coupled with one end (upwards directed, or downstream) to the pump cylinder (9) and which is supported at the housing (1) (lower part of the figure).

The inhalation device further comprises a longitudinal piston (5) with at least one reservoir-facing, or upstream, interior end (5A) which can be received in said cylinder (9). In other words, longitudinal piston (5) can be at least partially pushed into the pump chamber (9), resulting in a decrease of the interior volume of the pumping chamber (3). The term “interior volume” describes the volume of the space which extends from the reservoir-facing inlet of the pump cylinder (9) to the place where the interior end (5A) of the longitudinal piston (5) is located. In the depicted situation, the longitudinal piston (5) is almost entirely contained in the pump cylinder (9). As a result, the interior volume of the pumping chamber (3), situated between inlet valve (4) and the interior end (5A) of the longitudinal piston (5), is at a minimum.

Preferably, the section (or segment) of the pump cylinder (9) which serves as, or accommodates, the pumping chamber (3) and which receives the longitudinal piston (5) exhibits a circular inner cross-section whose diameter relatively closely (e.g. except for a small gap) matches the diameter of the circular outer cross-section of the corresponding segment of the longitudinal piston (5). Of course, other (e.g. non-circular) cross section shapes are possible as well.

According to the depicted embodiment, an inlet valve (4) is arranged between reservoir (2) and inlet of the pumping chamber (3) formed by the pump cylinder (9).

Furthermore, the inhalation device comprises a nozzle (6) which is connected liquid-tight to the exterior (or downstream) end (5B) of the longitudinal piston (5). The nozzle (6) is an impingement-type nozzle for generating the nebulized aerosol by collision of at least two liquid jets. Preferably, the cross sections of the liquid-containing channels are relatively small, typically in the region of microns.

Also depicted is an optional outlet valve (8) inside the longitudinal piston (5) for avoiding a backflow of liquid or air into the exterior end (5B) of the same from the outside. Outlet valve (8) is arranged in the interior end (5A) of the longitudinal piston (5). Liquid (F) can pass the outlet valve (8) in direction of the nozzle (6), but the outlet valve (8) blocks any undesired backflow in the opposite direction.

As can be seen in FIG. 1A, the longitudinal piston (5) is designed immobile with respect to the housing (1), and firmly attached to housing (1), indicated by the connection in the region of exterior end (5B) with housing (1). The longitudinal piston (5) is also firmly attached to the nozzle (6), which in turn is attached to housing (1) as well. In contrast, the pump cylinder (9) providing the pumping chamber (3) is designed to be moveable with respect to housing (1) and nozzle (6). The benefits of this design have been explained; reference is made to the respective sections of the description above.

FIG. 1B shows an inhalation device comprising a high-pressure medical pumping device according to the invention in the so-called primed position, ready to release the fluid upon user interaction. The pump cylinder (9) has been moved to its most upstream position, thereby loading the means for the exertion of a release force (7). Outlet valve (8) is closed due to negative pressure inside pumping chamber (3), and the inlet valve (4) is open towards the fluid reservoir (2). Increasingly collapsing walls of reservoir (2) allow the internal pressure in the reservoir (2) to remain nearly constant, while the pressure inside the pumping chamber (3) drops because of the propulsive longitudinal motion of the pump cylinder (9), thus increasing the volume of pumping chamber (3). As a result, the pumping chamber (3) has been filled with liquid (F) from the reservoir (2).

FIG. 1C shows a superposition of the ready state and primed state of a selected portion of an inhalation device comprising a pumping unit according to the invention. The figure illustrates the variable chamber height (CH) of the pump chamber by illustrating the different positions between ready and primed position. The figure further illustrates the relationship between outer diameter (OD) and inner diameter (ID) of the longitudinal piston. The figure is not to scale.

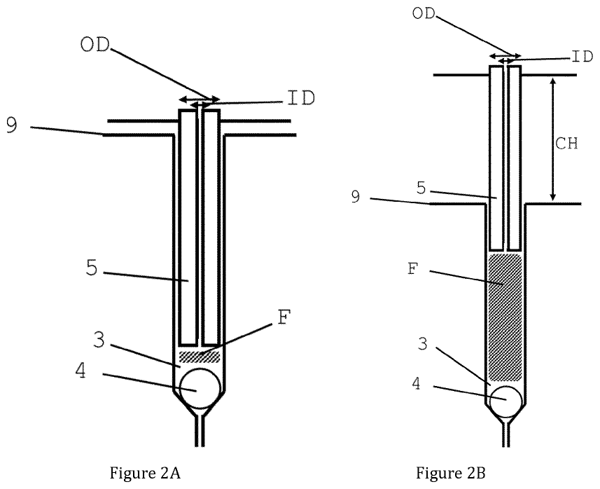

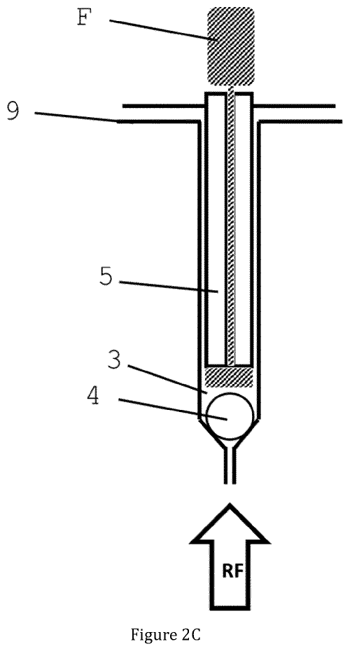

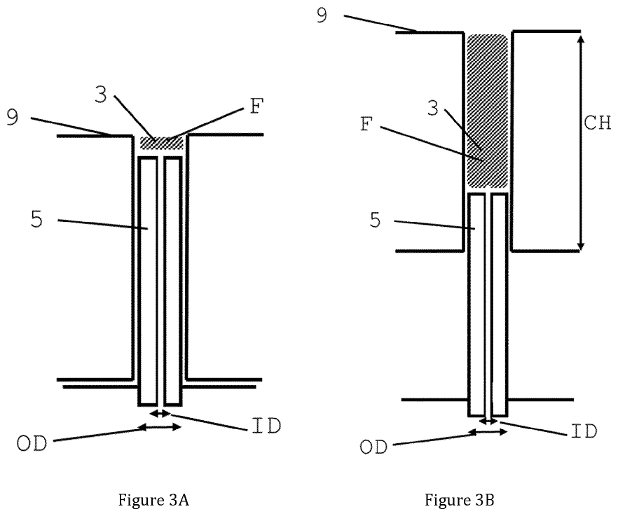

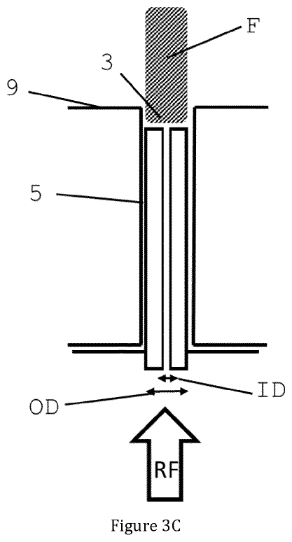

FIGS. 2A, 2B and 2C illustrate a pumping cycle of a pumping unit according to one embodiment of the invention in detail. The figures show a so-called moving chamber embodiment of the invention. FIG. 2A shows a pumping unit according to the invention in ready state. The pump chamber (3) may comprise a small amount of residual fluid (F) no released from the last cycle. The longitudinal piston has an outer diameter (OD) for which the pump cylinder (9) is adapted. The inner diameter defines a fluid connection to release the fluid (F), e.g., by connecting to a nozzle.

FIG. 2B shows the primed state, in which the pump chamber (3) has increased in size due to the movement of the pump cylinder (9). The movement distance corresponds to the variable chamber height (CH) which further defines the pump chamber and, in combination with the outer diameter (OD) of the longitudinal piston, defines the final volume of the pump chamber. The pump chamber (3) is filled with fluid (F).

FIG. 2C shows that after exertion of a release force on the pump cylinder (9), the cylinder is moved back into the rest position. The fluid (F) is released through the longitudinal piston (5) due to the release force (RF) exerted on the cylinder. The Fluid (F) is preferably transferred to a nozzle (not shown) and nebulized.

FIGS. 3A, 3B and 3C illustrate a pumping cycle of a further pumping unit according to one embodiment of the invention in detail. The figures show a so-called moving piston embodiment of the invention. FIG. 3A shows a pumping unit according to the invention in ready state. The pump chamber (3) may comprise a small amount of residual fluid (F) no released from the last cycle. The longitudinal piston has an outer diameter (OD) for which the pump cylinder (9) is adapted. The inner diameter defines a fluid connection to release the fluid (F), e.g., by connecting to a nozzle.

FIG. 3B shows the primed state, in which the pump chamber (3) has increased in size due to the movement of the longitudinal piston. The movement distance corresponds to the variable chamber height (CH) which further defines the pump chamber and, in combination with the outer diameter (OD) of the longitudinal piston, defines the final volume of the pump chamber. The pump chamber (3) is filled with fluid (F).

FIG. 3C shows that after exertion of a release force on the longitudinal piston (5), the piston is moved back into the rest position. The fluid (F) is released from the pump chamber due to the release force (RF) exerted on the piston. The Fluid (F) is preferably transferred to a nozzle (not shown) and nebulized.

### LIST OF REFERENCES

1 Housing
2 Fluid reservoir, reservoir
3 Pumping chamber
4 Inlet valve
longitudinal piston
5A Interior end
5B Exterior end
6 Nozzle
7 Means for exerting a release force
8 Outlet valve
9 pump cylinder
F Liquid, fluid
CH Variable Chamber Height
OD Outer Diameter
ID Inner Diameter
RF Release Force

### EXAMPLES

### Example 1

The inventors developed a hand-held inhalation device comprising a medical high-pressure pumping device according to the invention with the following parameters:

| | |
|---|---|
| Variable Chamber height (CH)/mm | 9.000 |
| Release Force (RF)/N | 60 |
| Outer Diameter (OD) longitudinal | 1.800 |
| piston/mm | |
| Inner Diameter (ID) longitudinal | 0.500 |
| piston/mm | |
| Pump chamber volume/mm³ | 22.902 |
| ratio RF/CH | 6.67 to 1 |
| ratio OD/ID | 3.6 to 1 |
| ratio RF/OD | 33.33 to 1 |
| Fluid pressure on release | 235.79 bar |
| spray duration/s | 2.8 |

The device as shown above provides a hand-held inhalation device for larger volumes, greater than 20 μL, which allows for a reasonable spray duration.

### Example 2

In another embodiment, a hand-held inhalation device comprising a medical high-pressure pumping device according to the invention with a spray volume of 25 μL provided a spray duration in experimental testing as follows:

| | |
|---|---|
| spray duration/s | 2.37 |

Theoretical calculations predict a spray rate of 10 μL/s, which was exceeded in the experimental data using a hand-held inhalation device comprising a medical high-pressure pumping device according to the invention with a spray volume of 25 μL.

1. A high-pressure medical pumping device for providing discrete portions of a medical fluid in pressurized form, wherein the pumping device comprises
a fluid inlet;
a fluid outlet; and
a pumping unit fluidically connecting the fluid inlet with the fluid outlet and being adapted to generate the discrete portions of the medical fluid in pressurized form and to convey the discrete portions of the medical fluid (in a downstream direction) from the fluid inlet to the fluid outlet;
wherein the pumping unit comprises
a pump cylinder comprising a pump chamber located within the pump cylinder comprising a fluid opening, wherein the pump chamber has a variable chamber height (CH); and
a longitudinal piston comprising a first end and a second end located opposite of the first end, wherein the longitudinal piston is at least partly located within the pump chamber of the pump cylinder such that at least the first end of the piston is located within the pump chamber, and wherein the piston and the pump cylinder are arranged for back and forth stroke movement of at least one of the piston and the pump chamber relative to each other, wherein the piston is of circular shape with an outer diameter (OD),
means to exert a release force (RF) to initiate a stroke movement of the piston and/or the pump chamber to convey the fluid to the fluid outlet;
wherein the piston comprises a fluid channel fluidically connecting the first end of the piston with the second end of the piston and being adapted to convey the medical fluid from the first or second end of the piston to the corresponding opposite end of the piston, wherein said fluid channel has a diameter corresponding to an inner diameter (ID) of the piston; and
wherein the pumping unit is adapted to provide the discrete portions of the medical fluid in pressurized form at a pressure of at least 100 bar.

2. The high-pressure medical pumping device according to claim 1, wherein the outer diameter of the piston is between 1.7 and 2.5 mm.

3. The high-pressure medical pumping device according to claim 1 or 2, wherein the inner diameter of the piston is 0.5 mm.

4. The high-pressure medical pumping device according to any of the preceding claims, wherein the release force is between 50 and 70 N.

5. The high-pressure medical pumping device according to any of the preceding claims, wherein the variable chamber height is between 6 and 12 mm.

6. The high-pressure medical pumping device according to any of the preceding claims, wherein the medical pumping unit is adapted to provide the discrete portions of the medical fluid in pressurized form at a pressure of at least 150 bar, or of at least 175 bar or of at least 200 bar.

7. The high-pressure medical pumping device according to any one of the preceding claims, wherein the pumping device is adapted to be comprised by an inhalation device, preferably by a hand-held inhalation device.

8. The high-pressure medical pumping device according to any one of the preceding claims, wherein the fluid outlet is fluidically connected to a nozzle for discharging and optionally aerosolizing the conveyed medical fluid in pressurized form, for example an impingement-type nozzle for aerosolizing the medical fluid by collision of at least two liquid jets of the medical fluid.

9. The high-pressure medical pumping device according to any one of the preceding claims, wherein the pump cylinder comprises a fluid inlet fluidically connected to fluid inlet of the pumping device (and optionally to the reservoir) and located downstream of the fluid inlet of the pumping device and wherein the second end of the piston is fluidically connected to the fluid outlet of the pumping device.

10. The high-pressure medical pumping device according to claim 9, wherein the piston is arranged in a fixed position within the pumping unit such that the piston is immobile during back-and-forth stroke movement of the pump chamber.

11. The high-pressure medical pumping device according to any one of claims 1 to 9, wherein the pump cylinder comprises a fluid outlet fluidically connected to the fluid outlet of the pumping device and located upstream of the fluid outlet of the pumping device and wherein the second end of the piston is connected to the fluid inlet of the pumping device.

12. The high-pressure medical pumping device according to claim 11, wherein the pump chamber is arranged in a fixed position within the pumping unit such that the pump chamber is immobile during back-and-forth stroke movement of the piston.

13. The high-pressure medical pumping device according to any one of the preceding claims, wherein the means to exert a release force on the pump chamber comprises a lockable means for storing potential energy when locked and for releasing the stored energy when unlocked, the means being arranged outside of and being mechanically coupled to at least one of the pump cylinder and the piston such that unlocking the means results in a propulsive longitudinal movement of at least one of the pump cylinder and the piston towards the corresponding other one of the pump cylinder and the piston.

14. The high-pressure medical pumping device according to any one of the preceding claims, wherein the ratio of the release force in N to the variable chamber height in mm is 6.6 to 6.7 to 1;
the ratio of the outer diameter of the longitudinal piston in mm to the inner diameter of the longitudinal piston in mm is 3.6 to 1; and
the ratio of the release force in N to the outer diameter of the longitudinal piston in mm is 33.3.

15. An inhalation device for the administration of a medical liquid in aerosolized form comprising a high-pressure medical pumping device according to any one of claims 1 to 14.
